(19) United States
(12) Patent Application Publication
(10) Pub. No.: US 2014/0248642 A1
(43) Pub. Date: Sep. 4, 2014

(54) APPARATUS AND METHOD FOR LATERAL FLOW AFFINITY ASSAYS

(71) Applicants: Anthony Edward George Cass, London (GB); Almudena Celaya Sanfiz, London (GB); Marian Rehak, London (GB)

(72) Inventors: Anthony Edward George Cass, London (GB); Almudena Celaya Sanfiz, London (GB); Marian Rehak, London (GB)

(73) Assignee: BIO NANO CONSULTING, London (GB)

(21) Appl. No.: 14/233,233

(22) PCT Filed: Jul. 19, 2012
(86) PCT No.: PCT/GB12/51733

§ 371 Date: Apr. 30, 2014

(30) Foreign Application Priority Data
Jul. 19, 2011 (GB) 1112395.7

(51) Int. Cl. G01N 33/543

(52) U.S. Cl. CPC G01N 33/54386; G01N 33/5438
USPC 435/7.92; 435/287.2

(57) ABSTRACT

The invention provides apparatus for the quantitative analysis of an analyte in a sample, comprising (i) a solid phase; and (ii) a detector, wherein the surface of the solid phase comprises (a)a first position for sample application, and (b)a second position, distant from the first position, wherein a first molecule that binds to the analyte and is capable of releasing a detectable species is either deposited at the first position or is added to the sample prior to application to the LF membrane, and wherein a second molecule that binds to the analyte is immobilised at the second position, and wherein an enzyme is immobilised, co-located with the immobilised molecule at the second position, and wherein the detector is located in close proximity to the immobilised molecule at the second position.

### BACKGROUND

Affinity assays are widely used in medical, food, and environmental research and are based on the principle of a reagent binding to a target analyte. The binding interaction is detected through some physico-chemical change, the magnitude of which is proportional to the analyte concentration. Many different reagents have been described including, but not limited to, antibodies, other proteins, nucleic acids, and synthetic receptors.

Antibodies are commonly used as the reagent and many different assay formats using antibodies have been described. These are collectively referred to as immunoassays. One type of immunoassay which is particularly suitable for point of care tests (POCTs) is the lateral flow (LF) immunoassay (also referred to as an immunochromatographic assay).

LF immunoassays are a well-established and robust technology for detection of antigens. They are adapted to operate along a single axis to suit a test strip format and typically employ a sandwich (also referred to as 2-site, reagent excess or non-competitive) format. In this format one of two antibodies is labelled, typically with coloured particles, and dried as a soluble preparation on a LF membrane (typically a hydrophobic nitrocellulose or cellulose acetate membrane). This first antibody dissolves in the sample, whilst a second antibody is fixed to the LF membrane some short distance from the first antibody. Sample solution carries the first antibody to the second through capillary flow and the immune complex that forms during this flow is captured by the second antibody forming a visible line. Excess labelled first antibody is carried past this ‘test line’ to react at a control line. This provides a qualitative read-out.

More recently the format has been modified such that the second antibody, labelled with biotin, is also soluble and the sandwich complex is formed in solution during the LF before being captured at a streptavidin line. US2010/0267166 describes a device for detecting an analyte, comprising a labelled conjugate comprising a binding member reacted with a first epitope of the analyte and a label, and a biotinylated capture component having a site reactive with a second epitope of the analyte.

It would be advantageous to adapt this assay format further, in order to increase its sensitivity and provide a quantitative numerical readout of the amount of immune complex at the second line, whilst retaining the essential simplicity and robustness of the LF format.

### SUMMARY OF THE INVENTION

According to a first aspect of the invention, apparatus for the quantitative analysis of an analyte in a sample comprises:

(i) solid phase; and

(ii) a detector,

wherein the surface of the solid phase comprises:

(a) a first position for sample application; and

(b) a second position distant from the first position,

wherein a first molecule that binds to the analyte and is capable of releasing a detectable species is either deposited on the solid phase or is added to the sample prior to application to the solid phase, and

wherein a second molecule that binds to the analyte is immobilised at the second position, and

wherein an enzyme is immobilised, co-located with the immobilised molecule at the second position, and

wherein the detector is located in close proximity to the immobilised molecule at the second position.

According to a second aspect of the invention, a method for quantitative detection of target molecules in a substrate-containing sample comprises:

a) dissolving a first soluble labelled binding molecule that is capable of releasing a detectable species and a second soluble labelled binding molecule in the sample to form a complex;

b) applying the complex formed in step a) to a solid phase, wherein the surface of the solid phase comprises:
i) a first position for application of the complex; and
ii) a second position, distant from the first position
wherein a molecule that binds to the second labelled binding molecule is immobilised at the second position, wherein an enzyme is immobilised co-located with the immobilised molecule at the second position, and wherein a detector is located in close proximity to the immobilised molecule at the second position; and

c) detecting the detectable species at the detector,

wherein detection of said species is proportional to the target molecule content of the sample.

According to a third aspect of the invention, the apparatus described in the first aspect of the invention is used in the method for quantitative detection of target molecules in a substrate-containing sample according to the second aspect of the invention.

### DESCRIPTION OF THE DRAWINGS

FIG. 1 is a schematic representation of the amplified electrochemical lateral flow (LF) test strip;

FIG. 2 is a schematic representation of the amplified electrochemical reaction;

FIG. 3 is a graphical representation of the pH change as a function of the enzymatic reaction between urease and urea in a 2 mM Tris/HCl buffer solution;

FIG. 4 is a graphical representation of the pH change as a function of the enzymatic reaction between pyrophosphate and pyrophosphatase in a solution with a low buffering capacity (2 mM Tris/HCl buffer with addition of 2 mM MgCl₂);

FIG. 5 is a graph showing the effect of increasing the local pH on release of a first encapsulated label;

FIG. 6 shows a similar effect to FIG. 5 but using potassium ferricyanate as the first label rather than ferrocene carboxylic acid; and

FIG. 7 shows the production of current as a function of ferrocene carboxylic acid release from polymeric bead at different time points.

### DETAILED DESCRIPTION OF THE INVENTION

The present invention addresses the aims of increasing the sensitivity of binding assays and is described in detail with reference to the lateral flow (LF) immunoassay. The invention provides a quantitative numerical readout of the amount of immune complex present in a sample, whilst retaining the simplicity and robustness of the assay format, by replacing the visual estimation of immune complex formation with an electrochemical measurement.

This is achieved, for example, by labeling one of two binding molecules, which may be antibodies, with particles that function as a source of a redox reagent that is detected at the test line. However, other approaches are also effective and these include, but are not limited to: encapsulated surface enhanced resonance Raman scattering (SERRS) active dyes; encapsulated mixture of a fluorescent molecule and a quencher; and encapsulated luciferase substrate to produce light emission at the test line. Therefore, although the invention is illustrated with respect to lateral flow assays, the scope of the invention is not limited to either the lateral flow assay format or to the antibody-antigen interaction detection.

According to a first aspect, the invention provides an apparatus for the quantitative analysis of an analyte in a sample, comprising:

(i) solid phase; and

(ii) a detector,

wherein the surface of the solid phase comprises:

(a) a first position for sample application; and

(b) a second position, distant from the first position,

wherein a first molecule that binds to the analyte and is capable of releasing a detectable species is either deposited on the solid phase or is added to the sample prior to application to the solid phase, and

wherein a second molecule that binds to the analyte is immobilised at the second position, and

wherein an enzyme is immobilised, co-located with the immobilised molecule at the second position, and

wherein the detector is located in close proximity to the immobilised molecule at the second position.

As used herein, the term “distant from” means that the second position is not immediately adjacent to the first position. Preferably, the second position is located downstream from the first position in the direct of flow.

As used herein, the term “close proximity” means that the detector must be positioned sufficiently close to the immobilized molecule at the second position to be able to detect any change in current or charge passed at said second position. Preferably, the detector is located as close as possible to the immobilized molecule at the second position.

The solid phase may be any suitable solid material or membrane, such as porous and/or non-porous surfaces such as silicon, silicon oxide, metallic and metal-coated surfaces, polymeric and polysaccharide surfaces. Preferably, the solid phase is a lateral flow (LF) membrane, preferably comprising chromatogenic media, which may be polymeric or cellulosic, such as hydrophobic nitrocellulose or cellulose acetate.

According to a second aspect, the present invention also provides a method for quantitative detection of target molecules in a substrate-containing sample. The method comprises the following steps:

a) dissolving a first soluble labelled binding molecule that is capable of releasing a detectable species and a second soluble labelled binding molecule in the sample to form a complex;

b) applying the complex formed in step a) to a solid phase, wherein the surface of the solid phase comprises:
i) a first position for application of the complex; and
ii) a second position distant from the first position
wherein a molecule that binds to the second labelled binding molecule is immobilised at the second position, wherein an enzyme is immobilised co-located with the immobilised molecule at the second position, and wherein a detector is located in close proximity to the immobilised molecule at the second position; and

c) detecting the detectable species at the detector,

wherein detection of said species is proportional to the target molecule content of the sample.

Preferably, the target molecule is an antigen and the first and second labelled binding molecules are antibodies. In such an embodiment, the complex formed is an immune complex.

Step (c) may take place as the complex is transported to the second position. Such transportation may be by means of capillary action, microfluidics or electrophoretic migration.

The apparatus described in the first aspect of the invention can be used in the method for quantitative detection of target molecules in a substrate-containing sample according to the second aspect of the invention.

The apparatus and method of the invention are exemplified by the combinations of enzyme and substrate shown in Table 1. However, many other combinations are also within the scope of the invention and would be apparent to one skilled in the art of enzymology.

TABLE 1

| Enzyme | Substrate |
|---|---|
| Urease | Urea |
| Glucose Oxidase | Glucose |
| Pyrophosphatase | Pyrophosphate |
| Alkaline phosphatase | Phenylphosphate |
| β-lactamase | Penicillin |

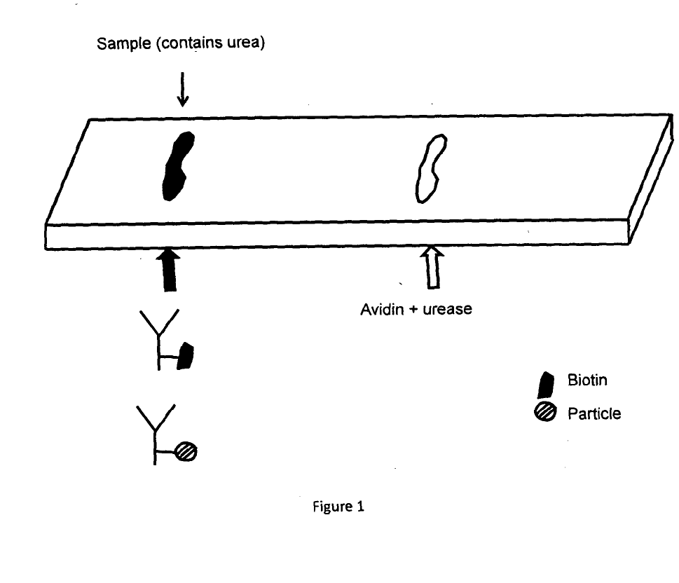

The method utilises the presence of substrate in the sample to change the local pH at the test line through the co-deposition of a corresponding enzyme immobilized at the second position. As would be understood by a person skilled in the art, the term “corresponding enzyme” to refer to an entity that catalysis the chemical reaction with a given substrate. Examples of compositions of the immune complexes that are applied to the solid phase membrane and the test line (i.e. the second position) are illustrated schematically in FIG. 1.

The first label that is capable of releasing a detectable species is preferably a redox probe, and is preferably encapsulated in a polymer. The polymer may be an enteric coating material which dissolves when subjected to a pH change, typically under alkaline conditions, or alternatively it may be a ‘smart’ stimuli responsive polymer that swells in response to a pH change.

Suitable enteric polymers include polyvinyl acetate phthalate, hydroxypropyl methylcellulose phthalate, methacrylic acid, cellulose acetate trimellitate, carboxymethyl ethylcellulose and hydroxypropyl methylcellulose acetate succinate.

Suitable pH-responsive ‘smart’ polymers include poly(propylacrylic acid) and chitosan.

Suitable redox probes undergo fast, preferably diffusion-limited, electron exchange at the detector and include complexes of iron, osmium, copper and ruthenium, as well as organic molecules such as flavins and dyes. Specific examples include but are not limited to: ferrocene and its derivatives; iron complexes such as hexacyanide; ruthenium complexes such as hexamine; osmium complexes as tris(bipyridyl); thiazine dyes; phenazine dyes; riboflavin and its derivatives; and tetrathiafulvalene and its derivatives. Additional examples will be apparent to those skilled in the art.

The second binding molecule is preferably labelled with biotin and the molecule that binds to it, and which is immobilised at the second position, is preferably avidin or streptavidin.

The detector located in close proximity to the immobilised molecule at the second position is preferably positioned below the test line (i.e. at or underneath the second position on the LF membrane). The detector is preferably an electrode, and preferably a screen printed electrode. The skilled person will be familiar with the term “screen-printed electrode”. However, for the avoidance of doubt, this is defined as a conducting carbon ink or metal paste film deposited on an inert support, such as PVC, ceramic, and alumina or polyester, and incorporating reference and counter electrodes. The electrode is preferably poised at a potential where there is a diffusion-limited reduction of the redox probe and a minimal background current from the sample. The detector must be positioned in sufficiently close proximity to the immobilized molecule at the second position (i.e. the test line) to be able to detect the change in current or charge passed.

The apparatus and method of the invention may be used for the in vitro quantitative analysis of many analytes including antigens, antibodies, other proteins and the products of nucleic acid amplification tests. The test sample may be selected from the following non-limiting group of body samples obtained from a subject or patient: urine; saliva; serum; plasma; whole blood; faeces; and exudates (e.g. from wounds or lesions). Alternatively, the sample may be a non-clinical material, such as soil, air, water or food matter.

The apparatus and method of the invention can be used as a tool to aid diagnosis and patient management. For example, the assay can be used to identify, confirm, or rule out disease in symptomatic patients, or to accurately prescribe therapeutic drugs and to monitor treatment, for example to monitor blood sugar levels in diabetic patients or to determine pregnancy. Other uses also include in epidemiology, where the rapid assay can be used to detect and monitor the incidence or prevalence of disease for targeting and evaluating health programs, as well as in screening to determine the prevalence of disease in asymptomatic individuals.

The terms ‘subject’ and ‘patient’ are used interchangeably herein and refer to a mammal including a non-primate (e.g. a cow, pig, horse, dog, cat, rat and mouse) and a primate (e.g. a monkey and human), and preferably a human.

By way of example, we illustrate an embodiment of the invention as a lateral flow immunoassay wherein the first and second binding molecules are antibodies, the target analyte is an antigen and the solid phase is a lateral flow membrane.

Upon addition of the substrate-containing sample, the two antibodies dissolve and form the immune complex which is carried by lateral flow to the test line. The immune complex travels with the liquid front and is captured at the test line by the reaction between the biotin component of the immune complex and avidin/streptavidin present in the test line. Once the substrate arrives at the test line there is a local change in pH due to the conversion of the substrate within the test sample. This may be a change to a more acidic environment (i.e. a decrease in pH) to a more alkaline environment (i.e. an increase in pH) depending upon the specific enzyme used.

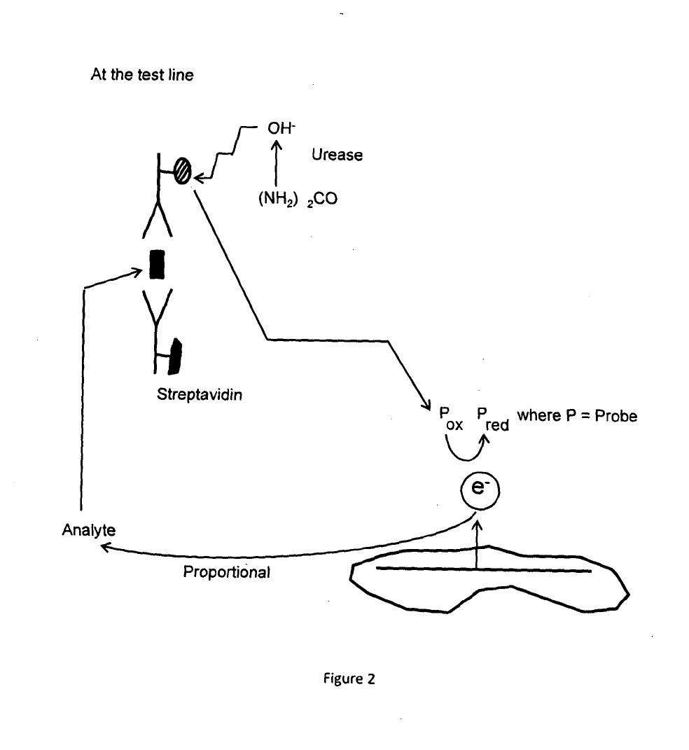

The amplified electrochemical reaction that takes place at the test line is illustrated schematically in FIG. 2.

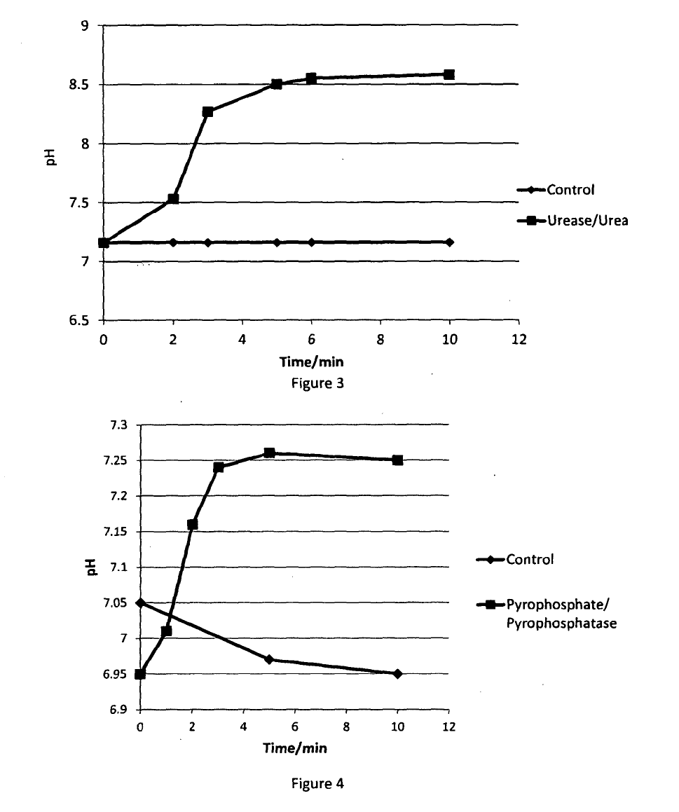

In one embodiment, the substrate-containing sample is preferably urine and the corresponding enzyme is therefore preferably urease. The concentration of urea in urine is around 10-20 times the Km value for urease; therefore the enzyme will be running at its maximal rate. The change in pH results in the redox probe undergoing reduction and releasing the redox species, which is detected from the current (or charge passed) at the underlying electrode. The current resulting from the released redox species is proportional to the antigen content of the sample. Therefore, by measuring the current (or charge passed) at the electrode, the antigen content of the sample can be determined quantitatively. FIG. 3 illustrates the pH change as a function of the enzymatic reaction between urease and urea in a solution with a low buffering capacity (2 mM Tris/HCl buffer).

Alternatively, the substrate-containing sample may be pyrophosphate and the corresponding enzyme is therefore preferably pyrophosphatase. FIG. 4 illustrates the pH change as a function of the enzymatic reaction between pyrophosphate and pyrophosphatase in a solution with a low buffering capacity (2 mM Tris/HCl buffer with addition of 2 mM MgCl₂).

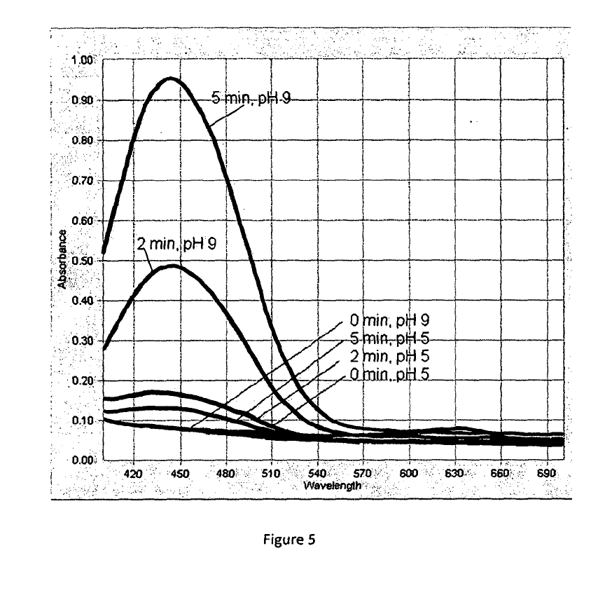

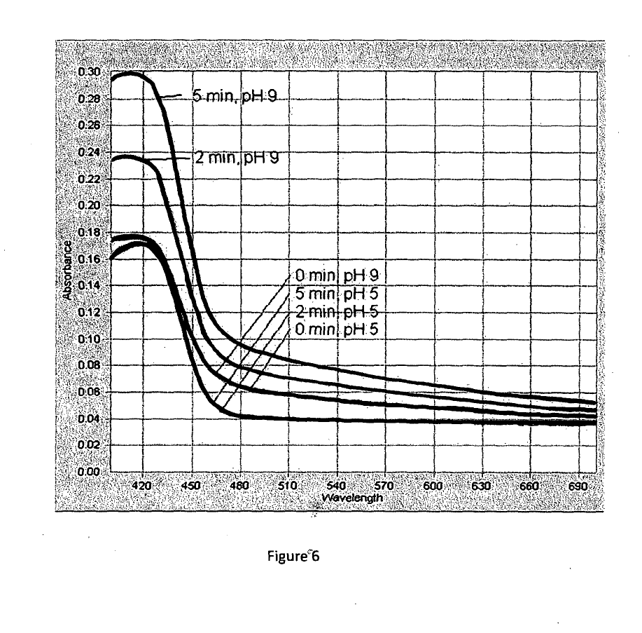

The effect of increasing the local pH on release of the first encapsulated label (a redox probe, specifically a ferrocene derivative) is illustrated in FIG. 5. This graph shows the spectrophotometric determination of ferrocene carboxylic acid release from polymeric beads as a function of time, at an alkaline pH of 9. As can be seen from the control values, when the pH is maintained at a mildly acidic pH of 5 the polymer does not dissolve and there is no increase in absorbance.

FIG. 6 illustrates a similar effect, however in this experiment the redox probe is potassium ferricyanate. The graph shows the spectrophotometric determination of release of the encapsulated probe from polymeric beads as a function of time, at an alkaline pH of 9. As can be seen from the control values, when the pH is maintained at a mildly acidic pH of 5 the polymer does not dissolve and there is no increase in absorbance.

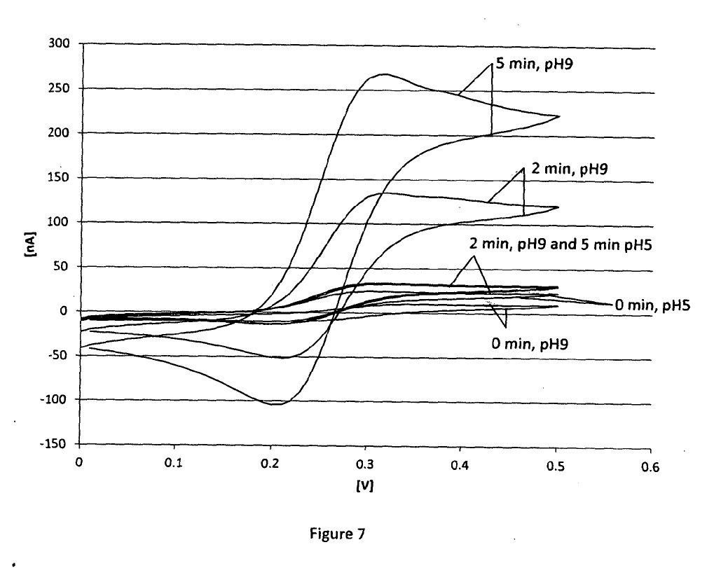

FIG. 7 illustrates the production of current as a function of the ferrocene carboxylic acid release from the polymeric beads at different time points and at an alkaline test pH of 9 and a mildly acidic control pH of 5. The graph shows that at pH 9 more electro-active molecules were released from the polymer beads as a function of polymer dissolution at the elevated pH. This release is reflected in the increase of the detected current. No current increase was observed at pH 5.

1. Apparatus for the quantitative analysis of an analyte in a sample, comprising
(i) a solid phase; and
(ii) a detector,
wherein the surface of the solid phase comprises:
(a) a first position for sample application; and
(b) a second position, distant from the first position,
wherein a first molecule that binds to the analyte and is capable of releasing a detectable species is either deposited at the first position or is added to the sample prior to application to the LF membrane, and
wherein a second molecule that binds to the analyte is immobilised at the second position, and
wherein an enzyme is immobilised, co-located with the immobilised molecule at the second position, and
wherein the detector is located in close proximity to the immobilised molecule at the second position,
wherein a substrate for the enzyme is either normally present in the sample, is added to the sample prior to application to the solid phase, or is deposited at the first position on the solid phase prior to sample application, and
wherein interaction between the enzyme and the substrate results in release of a detectable species from the first molecule.

2-3. (canceled)

4. Apparatus according to claim 1, further comprising means for converting the detection of said species by the detector into a value of concentration of analyte in the sample.

5. Apparatus according to claim 1, wherein the detector is an electrode and the detectable species is an encapsulated redox species.

6. Apparatus according to claim 1, wherein the enzyme is urease and the substrate is urea.

7. Apparatus according to claim 1, wherein the solid phase is a lateral flow membrane.

8. A method for quantitative detection of target molecules in a substrate-containing sample, comprising:
a) dissolving a first soluble labelled binding molecule that is capable of releasing a detectable species and a second soluble labelled binding molecule in the sample to form a complex;
b) applying the complex formed in step a) to a solid phase, wherein the surface comprises
i) a first position for application of the complex, and
ii) a second position, distant from the first position
wherein a molecule that binds to the second binding molecule is immobilised at the second position, wherein an enzyme is immobilised co-located with the immobilised molecule at the second position, and wherein a detector is located in close proximity to the immobilised molecule at the second position; and
c) detecting the detectable species at the detector as the complex is transported to the second position,
wherein detection of said species is proportional to the target molecule content of the sample.

9. A method according to claim 8, wherein the target molecule is an antigen.

10. A method according to claim 8, wherein the first binding molecule, the second binding molecule, or both are antibodies.

11. A method according to claim 10, wherein the first antibody is labelled with an encapsulated redox probe.

12. A method according to claim 10, wherein the second antibody is labelled with biotin and the molecule that binds to it and is immobilised at the second position is avidin.

13. A method according to claim 8, wherein step (c) comprises detecting the change in current or charge passed at the detector due to the change in local pH and resultant release of the redox probe.

14. A method according to claim 13, wherein the current resulting from oxidation or reduction of the redox probe is proportional to the antigen content of the sample.

15. A method according to claim 8, wherein the detector is an electrode.

16. A method according to claim 15, wherein the electrode is a screen printed electrode.

17. A method according to claim 8, wherein the redox probe is encapsulated in a polymer.

18. A method according to claim 17, wherein the polymer is an enteric coating which dissolves under alkaline conditions.

19. A method according to claim 17, wherein the polymer swells in acidic or alkaline conditions.

20. A method according to claim 8, wherein the sample is a urea-containing sample.

21. A method according to claim 8, wherein the enzyme is urease

22. (canceled)
